(19) United States
(12) Patent Application Publication
(10) Pub. No.: US 2017/0319074 A1
(43) Pub. Date: Nov. 9, 2017

(54) SYSTEM AND METHOD FOR PROVIDING AN INDICATION OF THE WELL-BEING OF AN INDIVIDUAL

(71) Applicant: Chee Seng Keith LIM, Singapore (SG)

(72) Inventor: Chee Seng Keith LIM, Singapore (SG)

(21) Appl. No.: 15/522,290

(22) PCT Filed: Oct. 15, 2015
(86) PCT No.: PCT/SG2015/050391

§ 371 Date: Apr. 27, 2017

(30) Foreign Application Priority Data
Oct. 28, 2014 (SG) 10201407018Y

(51) Int. Cl. A61B 5/00; G06F 19/00; A61B 5/16; A61B 5/024

(52) U.S. Cl. CPC A61B 5/0077; G06F 19/3431; G06F 19/345; A61B 5/02438; A61B 5/6898; A61B 5/165; G06F 19/3487

(57) ABSTRACT

Systems and methods for providing an indication of the well-being of an individual are disclosed. In particular, the systems and methods utilizes data obtained from physical sensors such as heartbeat monitors and social media platforms for deriving and/or assigning an indication of the individual's feelings or emotional well-being, are described.

### FIELD OF THE INVENTION

The present invention relates to a system and method for providing an indication of the well-being of an individual.

### BACKGROUND ART

The following discussion of the background to the invention is intended to facilitate an understanding of the present invention only. It should be appreciated that the discussion is not an acknowledgement or admission that any of the material referred to was published, known or part of the common general knowledge of the person skilled in the art in any jurisdiction as at the priority date of the invention.

Throughout the specification, unless the context requires otherwise, the word “comprise” or variations such as “comprises” or “comprising”, will be understood to imply the inclusion of a stated integer or group of integers but not the exclusion of any other integer or group of integers.

Furthermore, throughout the specification, unless the context requires otherwise, the word “include” or variations such as “includes” or “including”, will be understood to imply the inclusion of a stated integer or group of integers but not the exclusion of any other integer or group of integers.

With the advance in technology, mobile phones and wearable devices are increasingly used to provide a measure of well-being of an individual. An example is the use of mobile phone cameras and wearable watches for the detection and measurement of an individual's heartbeat.

Currently, analysis of an individual's heartbeat are however limited to determining an individual's physical well-being, such as whether there are any heartbeat irregularities, heart diseases, or high blood pressure, for example.

Increasingly, more people are aware of mental illnesses such as depression, and their impact or significant effect on peoples' behaviour, feelings and sense of mental or emotional well-being. The accurate and timely diagnosis of depression is a non-trivial task, since the common symptoms and the degree of depression can vary greatly from one person to another. This is particularly true in the early stages of depression when an immediate treatment typically promises the best results.

Various works have been proposed to estimate the likelihood that a person has (an onset of) depression using publicly available data reflecting certain user behaviour. For example, a study using social media platform shows that depressed people a. utilizes social media platform less than healthy people; b. are more likely to send social media messages late at night; c. uses an indicative vocabulary (words reflecting common symptoms and words reflecting emotions or feelings) and d. have a much smaller social network.

In light of the above, there exists a need to relate heartbeat information to an individual's emotional well-being or feelings in addition to physical well-being, without a need for the individual to verbalize how he/she is feeling when faced with external stimuli. There exists a further need to complement heartbeat information and other physical information with information obtained from an individual's social media platform to provide an overall indication of the well-being of the individual.

It is therefore an object of the invention to meet the above needs at least in part.

### SUMMARY OF THE INVENTION

The invention is suited (but not limited) to process heartbeat information of an individual for the purpose of relating the processed heartbeat information to the individual's sentiment or feelings. The correlation between the individual's heartbeat and sentiments is referred to as ‘Sentibeat™’ in the description. In an embodiment, assigned Sentibeats™ may be shared within a social network Hearti™ developed specifically for sharing the Sentibeats™ within a group of registered users, and/or across social networks such as Facebook™ or Twitter™, for example. In another embodiment, such sentiment may be used to detect whether an individual is in danger and if so information relating to the individual's location may be automatically routed to designated parties.

In accordance with an aspect of the invention there is a method for processing heartbeat information comprising the following steps: —obtaining a dataset of heartbeat information from an individual; deriving and/or calculating from the dataset of heartbeat information, at least one heartbeat index; and assigning an indication of the individual's feelings or emotional well-being based on the at least one heartbeat index.

Preferably, the heartbeat information includes at least one of the following: —number of heartbeats within a predetermined or stipulated duration; strength of the heartbeats recorded within the predetermined or stipulated duration; geographical location where the heartbeat data is obtained; sound file of the obtained heartbeats; time when the heartbeat data is obtained; and calendar events during the time when the heartbeat data is obtained.

Preferably, the at least one heartbeat index includes the highest number of heartbeats within the stipulated duration; the lowest number of heartbeats within the stipulated duration; the average number of heartbeats within the stipulated duration; and a plot of the heartbeat pattern.

Preferably, the indication of the individual's feelings or emotional well-being is based on a video, audio, word, emoticon, picture, or any combination thereof.

Preferably, the step of assigning the indication of the individual's feelings or emotional well-being is based on a combination of the heartbeat information and the at least one heartbeat index.

Preferably, the step of assigning the indication of the individual's feelings or emotional well-being is based on comparing the heartbeat information and heartbeat index with a previous assigned feeling or emotional well-being of the same individual.

In accordance with another aspect of the invention there is a system for processing heartbeat information comprising a heartbeat measuring device arrange to obtain a dataset of heartbeat information from an individual; a processor unit arranged in data communication with the heartbeat measuring device; the processor unit operable to derive or calculate from the obtained dataset of heartbeat information at least one heartbeat index; and assign an indication of the individual's feelings or emotional well-being based on the at least one heartbeat index.

Preferably, the heartbeat information includes at least one of the following: —number of heartbeats within a predetermined or stipulated duration; strength of the heartbeats recorded within the predetermined or stipulated duration; geographical location where the heartbeat data is obtained; sound file of the obtained heartbeats; time when the heartbeat data is obtained; and calendar events during the time when the heartbeat data is obtained.

Preferably, the at least one heartbeat index includes the highest number of heartbeats within the stipulated duration; the lowest number of heartbeats within the stipulated duration; the average number of heartbeats within the stipulated duration; and a plot of the heartbeat pattern.

Preferably, the indication of the individual's feelings or emotional well-being is based on a video, audio, word, emoticon, picture, or any combination thereof.

Preferably, the indication of the individual's feelings or emotional well-being is assigned based on a combination of the heartbeat information and the at least one heartbeat index.

Preferably, the indication of the individual's feelings or emotional well-being is assigned based on comparing the heartbeat information and heartbeat index with a previous assigned feeling or emotional well-being of the same individual.

In accordance with another aspect of the invention there is a non-transitory computer readable medium that stores software instructions such that when executed, the software instructions implement the method according to the first aspect of the invention.

In accordance with another aspect of the invention there is a system for providing an indication of the well-being of an individual comprising at least one physical sensor operable to sense a physical attribute of the individual; at least one social sensor operable to sense a social feature of the individual; and a feature extractor operable to extract a set of relevant information from the physical sensor and social sensor to form a feature vector or dataset for training and testing.

Preferably, the at least one physical sensor comprises a heartbeat sensor.

Preferably, the at least one social sensor comprises a social media account associated with the individual.

Preferably, the feature vector is trained via a machine learning algorithm to derive the indication of well-being of the individual.

Preferably, the feature vector is pre-processed before training takes place.

### BRIEF DETAILS OF THE DRAWINGS

In order that this invention may be more readily understood and put into practical effect, reference will now be made to the accompanying drawings, which illustrate preferred embodiments of the invention by way of example only, wherein:

FIG. 1 illustrates a high level flow diagram of the method for processing heartbeat information in accordance with an embodiment of the invention;

FIG. 2 is a block diagram of the system for processing heartbeat information in accordance with another embodiment of the invention;

FIG. 3 illustrates a logical flow chart of the use of the system of FIG. 2 for processing heartbeat information in accordance with an embodiment of the invention;

FIGS. 4a and 4b are examples of how the obtained heartbeat information may be stored and categorized into associated data file in accordance with an embodiment of the invention; and

FIG. 5 illustrates another embodiment of a system for processing information to provide an indication of an individual's mental and/or emotional well-being.

Other arrangements of the invention are possible and, consequently, the accompanying drawings are not to be understood as superseding the generality of the description of the invention.

### DESCRIPTION OF EMBODIMENTS OF THE INVENTION

In accordance with an embodiment of the invention there is a method 100 for processing heartbeat information comprising the following steps: —

a. obtaining a dataset of heartbeat information from an individual (step 120);

b. deriving or calculating from the dataset of heartbeat information, at least one heartbeat index (step 140); and

c. assigning an indication of the individual's feelings or emotional well-being based on the at least one heartbeat index (step 160).

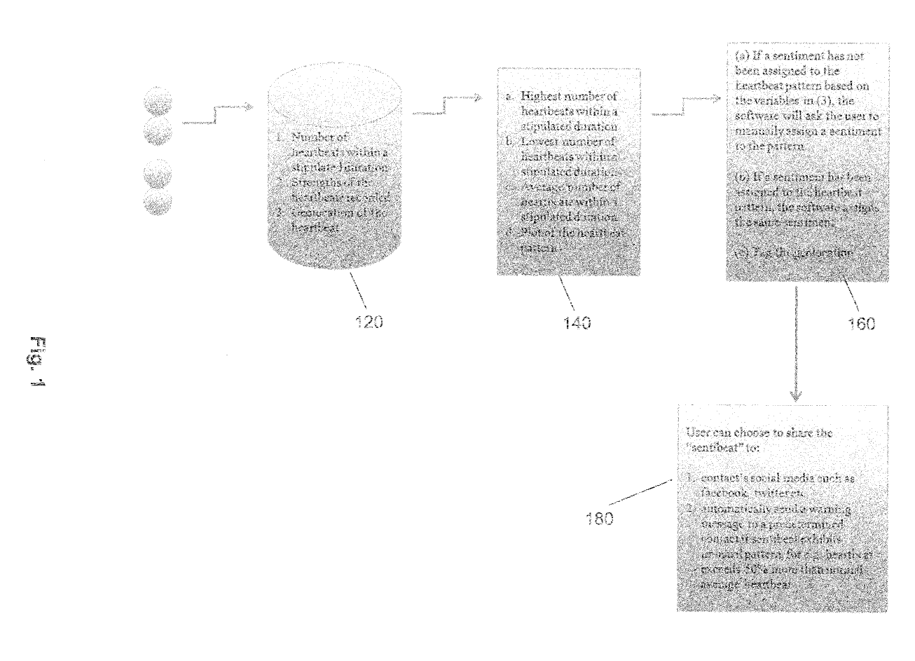

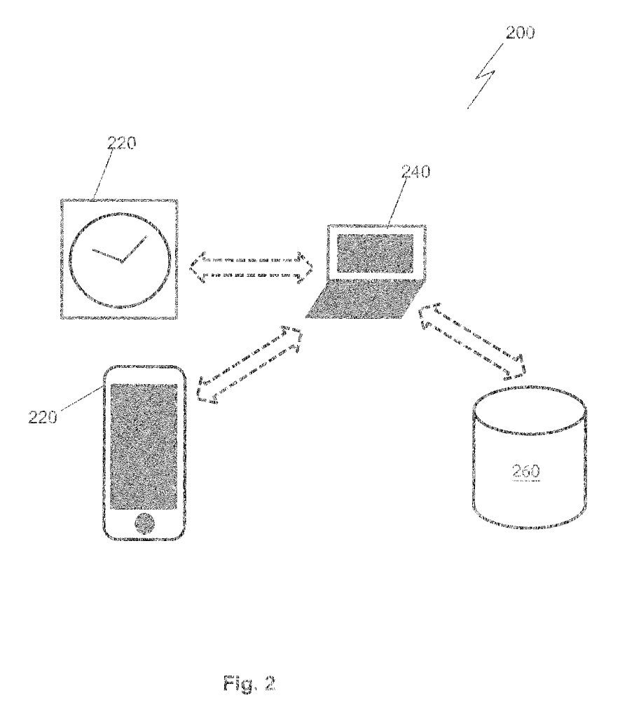

With reference to FIG. 1, the obtained dataset of heartbeat information comprise the following information: —

i. number of heartbeats within a predetermined or stipulated duration. The predetermined or stipulated duration may be, for example, within three seconds, six seconds, ten seconds intervals or longer;

ii. Strength of the heartbeats recorded within the predetermined or stipulated duration (strength may be calculated based on signal quality);

iii. Geographical location where the heartbeat data is obtained;

iv. Sound file of the obtained heartbeats;

v. Time when the heartbeat data is obtained; and

vi. Calendar events during the time when the heartbeat data is obtained, such calendar events may include concerts, carnivals, competitive tournaments, etc.

The obtained dataset of heartbeat information is then processed to derive or calculate at least one heartbeat index or heatbeat statistic. The statistics may include (but not limited to): —

the highest number of heartbeats within the stipulated duration;

the lowest number of heartbeats within the stipulated duration;

the average number of heartbeats within the stipulated duration (for example at every 60 seconds); and

a plot of the heartbeat pattern.

Upon deriving or calculating the heartbeat index, the heartbeat index is then used for providing a correlation between the heartbeat data with the individual's emotional well-being, feelings and/or sentiments (collectively referred to as ‘sentibeat’) for the specific period the heartbeat data is obtained. The correlation, which involves assignment of the sentibeat, may be based on one or more video, audio (music) files, word, emoticon, picture, or any multimedia file that is a combination of the above.

As an example, a sentibeat may be based on words such as ‘nervous’, ‘sad’, ‘excited’, ‘fearful’, ‘calm’, ‘angry’, or any other feelings entered by a user whose heartbeat information is assigned. In some embodiments, sentibeats may include quantitative measurements to form combinations and degree of emotions felt, for example 70% excited, 30% angry, for example. The user whose sentibeat is assigned ‘nervous’ may choose a video, music, an emoticon or picture to accompany the assigned word ‘nervous’. A multimedia file comprising a combination of the above may also be made available for the user to choose from.

The process of assigning a ‘sentibeat’ of the individual includes the step of comparing the heartbeat indices with an already populated database 260 of sentibeats provided by the same individual.

In the comparison process, if the ‘to-be-assigned’ sentibeat resembles an existing ‘sentibeat’ (i.e. for example 80% or more similarity), then it will be assigned to the existing sentibeat, such as ‘angry’. In determining the level of similarity between the existing sentibeat and the ‘to-be-assigned’ sentibeat, any standard correlation comparison techniques as known to a skilled person may be used.

If the ‘to-be-assigned’ sentibeat does not resemble any sentibeat in the populated database (i.e. for example less than 80% similarity), then there is a need for the user or individual to specify his/her feelings or sentiments manually.

One way to populate the ‘sentibeat’ database is through an iterative learning process, wherein the method correlate the heartbeat indices to one or more ‘sentiments’ based on an input of a user. The method learns or correct the correlated mappings based on updated information. As an example, a relatively large number of heartbeats with a high strength associated may either be assigned as an ‘exciting’ sentibeat or a ‘fear’ sentibeat. To further distinguish between ‘exciting’ or ‘fear’, heartbeat information such as geographical location, time of the day, and/or calendar event during which the heartbeats were recorded may be utilized. For example, a sentibeat of ‘exciting’ maybe assigned when the method 100, implemented in the form of a dedicated software application (colloquially known as ‘app’) on the individual's mobile device reads that the individual is watching a ‘Tennis tournament’ (based on his mobile device calendar event) at a stadium (based on his Geographical location information extracted on Google™/Apple™ maps) when the heartbeat pattern is recorded. Similarly, a sentibeat of ‘fear’ maybe assigned when the software ‘app’ reads that the individual is at a ‘Horror movie preview’ (based on his mobile device calendar event) at 2300 hours (based on the time captured when the heartbeat are recorded). If the software is not able to assign a ‘exciting’ or ‘fear’ sentibeat due to lack of data, the user will be prompted to predetermine his/her preference settings, e.g. always choose ‘exciting’ over ‘fear’, or the user may be prompted to enter a sentibeat for future classification.

In one embodiment, the detected heartbeats and/or generated ‘sentibeat’ may be utilized as a safety measure. In particular, if a heatbeat statistic such as the average number of heartbeats within the stipulated duration exhibits an unusual pattern, i.e. if heartbeat exceeds 50% more than the normal average heartbeat of the individual, then a warning message may be generated and sent to one or more persons related to the individual.

For example, if a young child is wearing a wearable device having method 100 as software instructions installed thereon, when the child meets with some form of danger and is unable to articulate himself/herself, his/her heartbeat will respond by beating much faster and stronger, thereby falling into the category of ‘unusual pattern’. In such cases, the installed software would sound an alarm and send a warning message (with the child's geographical location) to at least the parents and/or caregivers as predetermined during the setup of the wearable device.

In some embodiments, once a sentibeat has been assigned, the user or individual may opt to share or display this information over one or more social networks, and/or store the information for his future reference (step 180).

In accordance with another embodiment of the invention there comprises a system 200 for processing heartbeat information. The system 200 comprises a heartbeat receiver or measurement device 220 operable to obtain heart beat information over a predetermined duration from an individual. The heartbeat information is received in the form of a dataset comprising

i. number of heartbeats within a predetermined or stipulated duration. The predetermined or stipulated duration may be, for example, within three seconds, six seconds or ten seconds intervals;

ii. Strength of the heartbeats recorded within the predetermined or stipulated duration (based on signal quality);

iii. Geographical location where the heartbeat data is obtained;

iv. Sound file of the obtained heartbeats;

v. Time when the heartbeat data is obtained; and

vi. Calendar events during the time when the heartbeat data is obtained, such calendar events may include concerts, carnivals, competitive tournaments, etc.

The obtained dataset of heartbeat information is sent to a processor 240 for further processing. The processor 240 is operable to implement steps 140 and 160 as described in the previous embodiment for purpose of assigning or classifying a sentibeat of an individual.

The heartbeat receiver or measurement device 220 may be any device capable of measuring heartbeats from an individual. In one embodiment, the measurement device 220 may be a mobile phone having camera flash capabilities installed thereon to obtain heart beat measurements and information once an individual's finger is placed on the flash of the mobile phone.

Advantageously, the heartbeat receiver or measurement device 220 may be a wearable device such as a wearable watch having heartbeat sensors positioned on the wearable device for obtaining an individual's heartbeat data passively. Such devices may work without the need for an individual to actively position a part of his or her body (e.g. finger) onto the measurement device 220.

The processor 240 may be any computing device capable of processing electronic data. Advantageously, the processor 240 may be integrated with the measurement device 220 for the reduction of form factor.

The processor 240 may be in data communication with a database 260 or repository containing assigned Sentibeat™ data of the individual for the purpose of reference for the classifying or assignment of any new heartbeat information received from the individual.

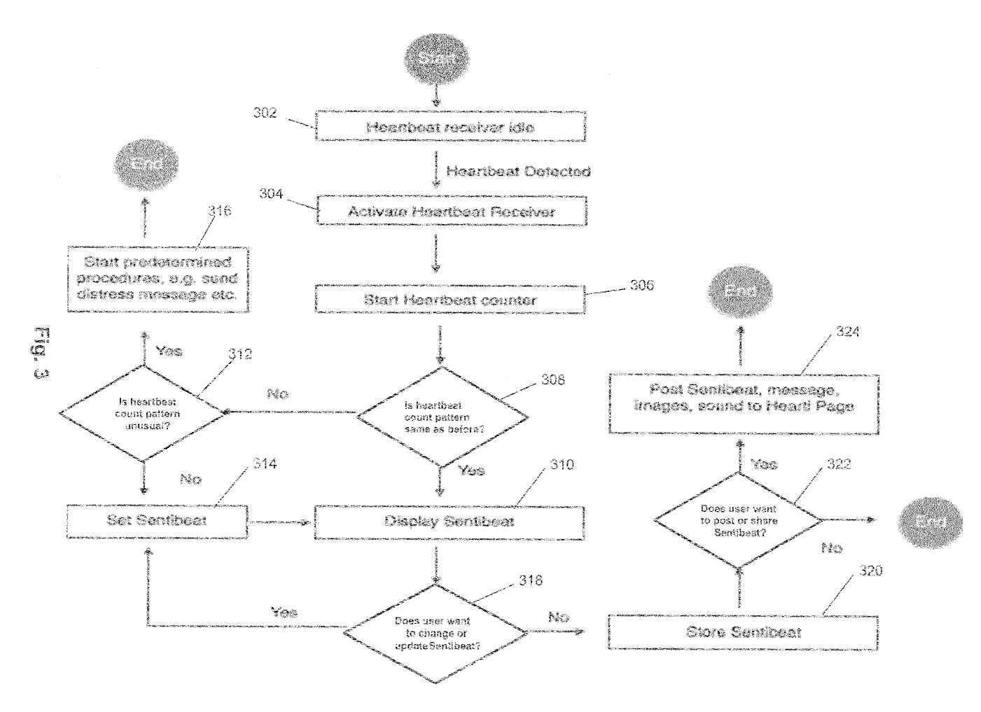

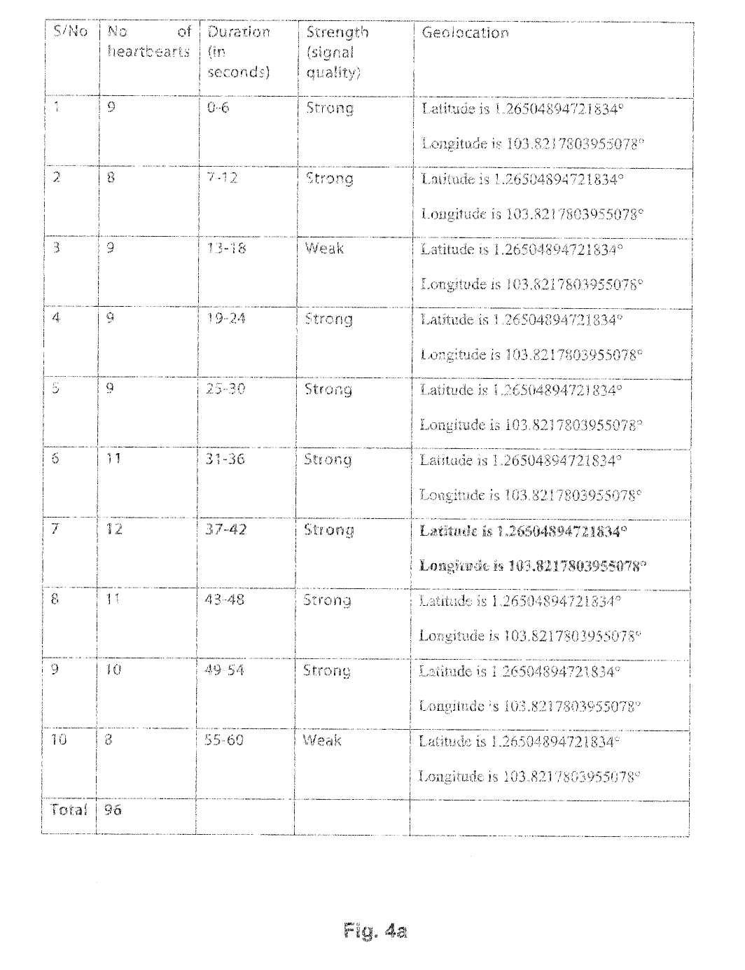

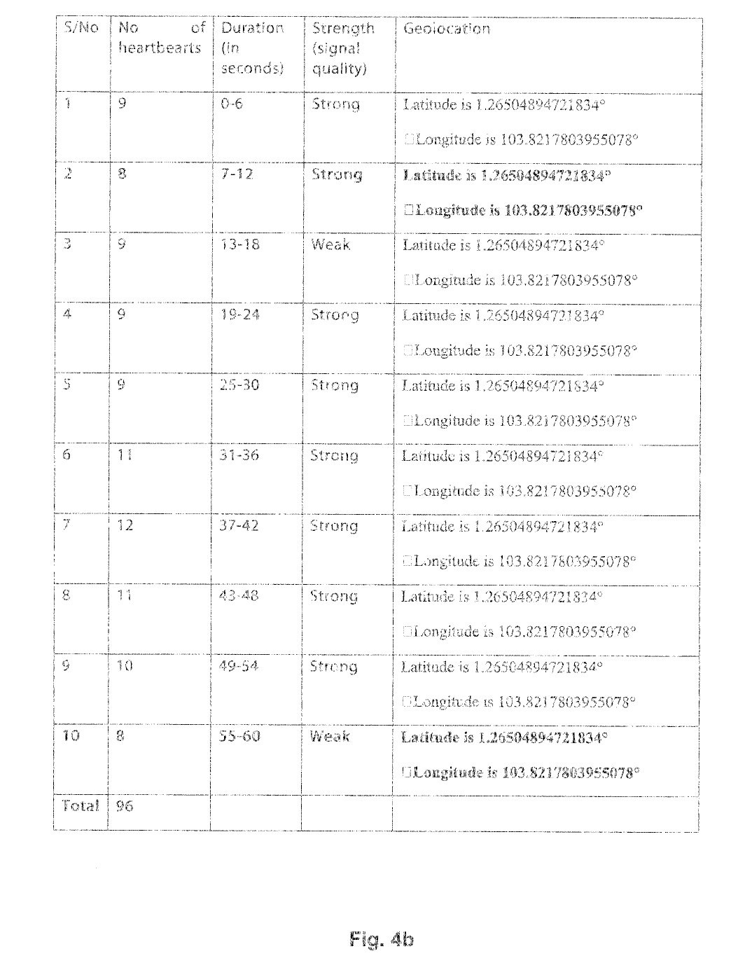

With reference to FIG. 3, in use, the heartbeat receiver 220 is idle (step 302), until heartbeat of an individual is detected, upon which the heartbeat receiver will ‘wake’ and be activated (step 304). Once activated, the heartbeat receiver 220 will begin to obtain heartbeat measurements (step 306). Upon receiving the heartbeat measurements for a predetermined period of time or duration, the relevant dataset will be obtained, the heartbeat indices generated and compared with the populated database (step 308). If the populated database comprises a similar heartbeat pattern (i.e. 80% or more similarity) as an already assigned sentibeat, then the obtained heartbeat measurements will be assigned to the sentibeat and displayed (step 310). If however, after comparison in step 308, if the heartbeat count or heartbeat index statistic is deemed to be different from all other entries in the ‘sentibeat’ database, then the heartbeat information will be further checked to determine if the heartbeat pattern is unusual (step 312) as described in the earlier embodiment. If it is determined that the heartbeat pattern is not unusual, then the individual will be requested/prompted to set his sentiment/feelings for correlation with his obtained heartbeat information (step 314). If the heartbeat pattern is deemed to be unusual, then the procedure of sending warning messages to one or more persons related to the individual will be initiated (step 316).

Upon display of the sentibeat (step 310), the user/individual will be prompted whether he/she wishes to change or update the displayed sentibeat (step 318). If so, he will be brought to step 314. Otherwise, the assigned sentibeat will be stored (step 320) for future heartbeat information to be compared against. The user may further be prompted on whether he/she would like to share or post the sentibeat (step 322) on one or more social medium and if not, the process ends. If the user wishes to post or share his sentibeat, then he would be brought to another user interface where he may select from a plurality of social media platform to post/upload the sentibeat onto (step 324).

In another embodiment of the invention there comprises a non-transitory computer readable medium that stores software instructions such that when executed, the executed software instructions implement the method 100 for processing heartbeat information by activating the heartbeat receiver to be in the idle mode until heart beat information is received from an individual, after which the steps 120, 140, 160 and/or 180 are executed. The non-transitory computer readable medium may be embedded within the wearable device or heartbeat monitor.

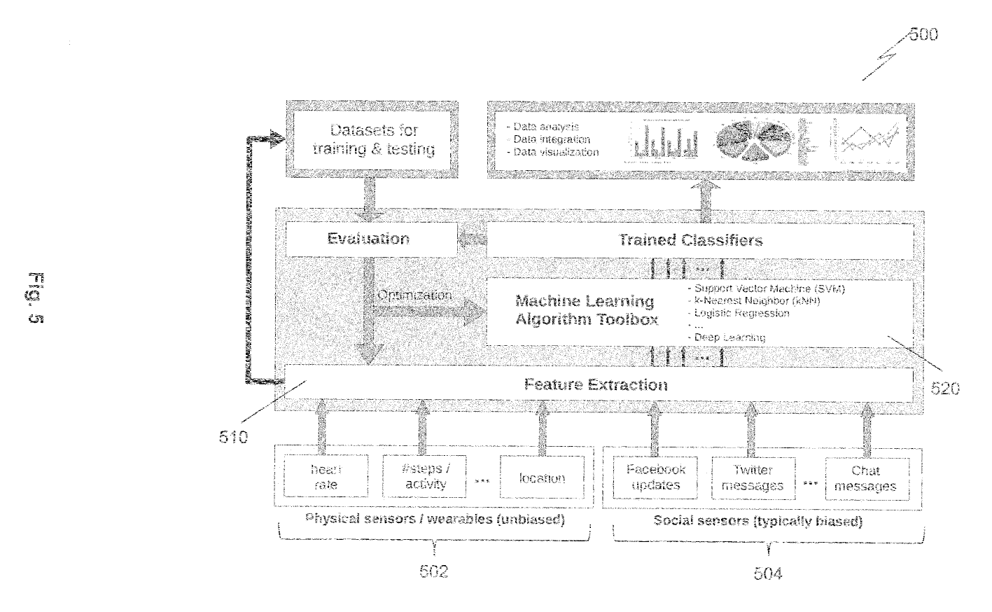

The above described embodiments relating to system and method for processing heartbeat information may be complemented or integrated with other information for deriving an indication of the mental or emotional well-being of an individual. As illustrated in FIG. 5, which forms yet another embodiment, there comprises a system 500 for processing information to provide an indication of an individual's mental and/or emotional well-being.

The system 500 comprises at least one physical sensor 502 and at least one social sensor 504, the physical sensor 502 and social sensor 504 in data communication with a feature extractor 510. The feature extractor 510 is operable to extract a set of relevant features or information from the physical sensor 502 and social sensor 504 for purpose of forming a feature vector or dataset for training and testing. The feature extractor 510 is in data communication with one or more processors 520 for purpose of training and classifying the dataset in order to derive one or more indicators of the mental or emotional well-being of the individual. The processor 520 may comprise the populated database 260 for purpose of training and classifying the feature vector.

The at least one physical sensor 502 is operable to sense a physical attribute of the individual. The physical sensor 502 may include heartbeat sensors, heartbeat monitors, and or other wearable devices having location based sensors such as GPS sensors, movement sensors such as gyroscope, and imaging sensors such as camera, or combinations of any of the above. The at least one physical sensor 502 is operable to measure one or more of an individual's physical attribute such as, but not limited to heartbeat, temperature, activity level, geographical location, etc.

The at least one social sensor 504 includes personal information extractable from the individual's social media platform such as Facebook™, and/or Twitter™. The social sensor may further include calendar events relating to the individual's activities or public events that are not specific or non-personal to the individual. The extractable information that form sensor values will thus include Tweets™, Facebook™ status messages and/or entries in the calendar. Social sensor 504 may also include the websites or Uniform Resource Locator (URL) accessed by the individual, the frequency of visits or any comments the individual posted on any social media in response to the status message of others.

For purpose of training and eventually classifying the feature dataset, the one or more processors 520 may comprise a machine learning module 522 for evaluating, optimizing and classifying the feature dataset. The machine learning module 522 may also include algorithms for processing and training the feature vector as input for obtaining an indication of the well-being of the individual as output. Such algorithms include support vector machine (SVM), k-nearest neighbour (k-NN) algorithm, logistic regression, deep learning and/or other combinations or supervised learning based algorithms known to a skilled person.

Collection of data from the at least one physical sensor 502 and the at least one social sensor 504 may be done via a dedicated software application (colloquially known as an ‘app’) installed on the mobile or wearable device such as a smart phone, smart watch. The app may be installed on a non-transitory computer readable medium and comprise the necessary logic for extracting the feature vector as will be further elaborated.

The embodiment will be next described in the context of its operation.

Information from the at least one physical sensor 502 and at least one social sensor 504 are selectively extracted to form a set of feature vector. The feature vector should be meaningful to the context of the type of well-being of the individual the system seeks to provide an indication of.

The feature vector specifies the set of features used for the learning and/or training process. A feature is a higher-level characteristic describing the input data. To illustrate the same, feature information in relation to a social sensor such as a Tweet™ may be

i. the number of followers;

ii. the number of sent tweets;

It is important to identify a “good” set of features for training so that there is correlation between the feature vector (input) and the indication of well-being (output) to be derived.

As a counter-example, the zodiac sign of a Twitter user is unlikely to be a useful feature to decide whether the users is emotionally well or is at risk of depression.

In addition, it is important to consider an optimum amount of features/information as the consideration of too many features may increase the risk of over-training classifiers—that is, the classifiers perform well on the training and test data but are not general enough to also perform well on new, unknown data. In addition, the number and types of features affect the system's performance in turns of runtime for the feature extraction and learning process. A case example of relevant information obtain from the physical sensor and social sensor associated with an individual (say Samantha) may be as follows: —
(i) Three used words, including emotion/feeling words identified: “Singapore”, “So excited”, “See cashof14”;
(ii) Number and type of used picture and/or character such as emoticon or emojis: 4;
(iii) Time and frequency of messages (a person sends and receives an average of 88 messages in a day;
(iv) Actual heartbeat patterns at a time: e.g. 90 beats per minute;
(v) Calendar events at that time: “Meeting with BOSS”
(vi) Geospatial information at that time: “Office”

As an example of an indicator of mental well-being, depression and other mental illnesses that affect users' well-being are unlikely to be associated with clear a straightforward patterns. It is thus important to select feature vectors that are accurate to derive the indicator of well-being. This includes that it is not obvious which types of features are most useful to detect these patterns—note that “bad” features will negatively affect the learning process by potentially detecting misleading patterns.

An advantage associated with the system 500 lies in the meaningful integration of the different types of sensor data (both physical and social) to find useful patterns and correlations. For example, while the heart rate, obtained from heart rate sensor(s) of an individual in itself may not be a very useful measure, in combination with social sensor information about that individual's activity level or what he or she writes on Twitter™ or Facebook™, the heart rate can be a powerful means to assess or re-assess the sentiment of, for example, a social network message (such as a tweet) obtained from one of the social sensor. The different types of collected data and the multi-dimensional nature of the obtained data require a variety of different data analysis tools for obtaining a feature vector for training. Such analysis may be regarded as a ‘pre-training’ analysis or input data analysis before training takes place. Such ‘pre-training’ analysis include, but are not limited to the following: —

(a) Time series analysis—More often than not, social sensor data such as the number of posted social messages (e.g. Tweets™) or the number of used happy/sad emotional icons (‘emojis’) is a useful indicator but may change over time. Monitoring such changes over a period of time can elicit longer-term trends as well as sudden spikes which turn can be correlated with events or changes in one person's environment.

(b) Social network analysis—Social network analysis relates to how deeply an individual is embedded in the social graph, which may be an indicator of his or her social engagement. Such analysis includes simple metrics such as the number of friends (or followers on Twitter), and also metrics that require more sophisticated network analysis techniques to calculate the similarity between nodes in a social network. For example, a depressed person is often likely to interact with other depressed people. Beside such network structure, social network analysis also investigates how information flows within the network, i.e., if and how people reply or forward messages from others.

(c) Linguistic analysis—Such analysis include the length of whole social messages; the length of individual sentences; and the used vocabulary. This also includes analysing if the social messages of a person are, for example, mostly ego-centric, i.e., with the person talking about him- or herself—which may be a phenomenon often observed in depressed people. Linguistic analysis is part of the wider research are of Natural Language Processing (NLP).

(d) Sentiment analysis—may be related to linguistic analysis, but often considered as a task of its own is sentiment analysis. Most commonly, sentiment analysis is treated as a NLP task evaluating the usage of sentiment-carrying words or other text tokens such as text emoticons, ‘emojis’ but also punctuation.

While data extracted from both physical and social sensors may often be noisy, physical sensors, compared to the individual's social sensor value may yield relatively unbiased measurements. In addition, although personalized data associated with each individual is utilized, public data such as information about current happenings (e.g., festivals, public holidays) or weather data, which potentially affect the mood of individuals, may further be utilized.

As case examples, there comprise various approaches for pre-processing social information extracted from the social sensor(s). For example, words within a lexicon (dictionary) may be assigned a sentiment score depending on the type of indicator of well-being to be derived. This may be in the form as shown in Table 1 below, which shows examples of words beginning with the alphabet ‘a’: —

TABLE 1
Examples of words and scores beginning with the alphabet ‘a’

| Word/phrase | Score |
|---|---|
| Adventure(s) | 2 |
| Adventurous | 2 |
| Affected | −1 |
| Affection | 3 |
| Affectionate | 3 |
| Afflicted | −1 |
| Affronted | −1 |

Variants for each word or phrase may be created to account for typographical error(s). These are input to take into account colloquial, spelling errors, and/or symbols inserted within/in combination with words or phrases which are increasingly common in social network messages, especially among the young.

Another approach that may be utilized in conjunction and supplement the aforementioned lexicon approach would be to utilize a publicly available dataset/database of keywords such as Tweets™ as a database for classification. Such publicly available database of tweets is already annotated and the vocabulary in the lexicon or dictionary may be compared with the words in the database.

It is to be appreciated that different feature sets, learning algorithms, and data cleaning/preprocessing steps may be utilized and combined. In particular, the sentiment score derived can be a new feature used for the classification database.

It is to be appreciated that the analysis of social sensors to provide a glimpse of an individual's mental well-being (i.e. human analytics) involves certain challenges due to element of subjectivity and variation across different fields. In particular there are two main challenges. Firstly, there comprises a wide variety of related but distinct research areas. Secondly, the core task in these areas typically do not allow for the straightforward implementation of algorithms that return the required results. The common scheme among these task is the identification of patterns within the data derived from peoples' individual or collected behaviour. The common solution to solve such problems is Machine Learning (ML). In a nutshell, ML algorithms work on large sets of data to uncover underlying patterns and processes. For example, given enough behavioural data from a large sample of people suffering from depression as well as from healthy people, supervised learning algorithms aim to identify the characteristics in the data that are most indicative for each group. Then, given the behavioural data of an unknown person, the machine learning algorithm can predict whether the person is likely to be healthy or likely to suffer from depression as an indication of well-being or non well-being.

It is to be understood that the above embodiments have been provided only by way of exemplification of this invention, and that further modifications and improvements thereto, as would be apparent to persons skilled in the relevant art, are deemed to fall within the broad scope and ambit of the present invention described herein. In particular, while the embodiments have been described in the context of the processing and classifying of an individual's heartbeat information, the invention may easily be extended to classify multiple individuals, each individual having an account with the database 260. In such a case, the database 260 also functions as an account database.

It would be further appreciated that although the invention covers individual embodiments, it also includes combinations of the embodiments discussed. Therefore, features described in one embodiment not being mutually exclusive to a feature described in another embodiment may be combined to form yet further embodiments of the invention.

1. A method for processing heartbeat information comprising the following steps: —
a. obtaining from a heartbeat measuring device a dataset of heartbeat information from an individual, the heartbeat information comprises a heartbeat data of the individual comprising a number of heartbeats within a predetermined or stipulated duration;
b. deriving and/or calculating from the dataset of heartbeat information by a processor unit, at least one heartbeat index; and
c. assigning an indication of the individual's feelings or emotional well-being based on the at least one heartbeat index;
wherein the heartbeat information includes a geographical location where the heartbeat data of the individual is obtained, and wherein if the indication of the individual's feelings or emotional well-being cannot be assigned based on the at least one heartbeat index, the geographical location is utilized to distinguish between a plurality of possible indications of the individual's feelings or emotional well-being.

2. The method according to claim 1, wherein the heartbeat information further includes at least one of the following: —strength of the heartbeats recorded within the predetermined or stipulated duration; sound file of the obtained heartbeats; time when the heartbeat data is obtained; and calendar events during the time when the heartbeat data is obtained.

3. The method according to claim 1, wherein the at least one heartbeat index includes the highest number of heartbeats within the stipulated duration; the lowest number of heartbeats within the stipulated duration; the average number of heartbeats within the stipulated duration; and a plot of the heartbeat pattern.

4. The method according to claim 1, wherein the indication of the individual's feelings or emotional well-being is based on a video, audio, word, emoticon, picture, or any combination thereof.

5. The method according to claim 2, wherein the step of assigning the indication of the individual's feelings or emotional well-being is based on a combination of the heartbeat information and the at least one heartbeat index.

6. The method according to claim 1, wherein the step of assigning the indication of the individual's feelings or emotional well-being is based on comparing the heartbeat information and heartbeat index with a previous assigned feeling or emotional well-being of the same individual.

7. A system for processing heartbeat information comprising
a heartbeat measuring device arranged to obtain a dataset of heartbeat information from an individual, the heartbeat information comprises a heartbeat data of the individual comprising a number of heartbeats within a predetermined or stipulated duration;
a processor unit arranged in data communication with the heartbeat measuring device; the processor unit operable to derive or calculate from the obtained dataset of heartbeat information at least one heartbeat index; and assign an indication of the individual's feelings or emotional well-being based on the at least one heartbeat index;
wherein the heartbeat information includes a geographical location where the heartbeat data of the individual is obtained, and wherein if the indication of the individual's feelings or emotional well-being cannot be assigned based on the at least one heartbeat index, the geographical location is utilized to distinguish between a plurality of possible indications of the individual's feelings or emotional well-being.

8. The system according to claim 7, wherein the heartbeat information further includes at least one of the following: —strength of the heartbeats recorded within the predetermined or stipulated duration; sound file of the obtained heartbeats; time when the heartbeat data is obtained; and calendar events during the time when the heartbeat data is obtained.

9. The system according to claim 7, wherein at least one heartbeat index includes the highest number of heartbeats within the stipulated duration; the lowest number of heartbeats within the stipulated duration; the average number of heartbeats within the stipulated duration; and a plot of the heartbeat pattern.

10. The system according to claim 7, wherein the indication of the individual's feelings or emotional well-being is based on a video, audio, word, emoticon, picture, or any combination thereof.

11. The system according to claim 8, wherein the indication of the individual's feelings or emotional well-being is assigned based on a combination of the heartbeat information and the at least one heartbeat index.

12. The system according to claim 7, wherein the indication of the individual's feelings or emotional well-being is assigned based on comparing the heartbeat information and heartbeat index with a previous assigned feeling or emotional well-being of the same individual.

13. A non-transitory computer readable medium that stores software instructions such that when executed, the software instructions implement the method according to claim 1.

14. A system for providing an indication of the well-being of an individual comprising: —
at least one physical sensor operable to sense a physical attribute of the individual;
at least one social sensor operable to sense a social feature of the individual; and
a feature extractor operable to extract a set of relevant information from the physical sensor and social sensor to form a feature vector or dataset for training and testing.

15. The system according to claim 14, wherein the at least one physical sensor comprises a heartbeat sensor.

16. The system according to claim 14, wherein the at least one social sensor comprises a social media account associated with the individual.

17. The system according to claim 14, wherein the feature vector is trained via a machine learning algorithm to derive the indication of well-being of the individual.

18. The system according to claim 14, wherein the feature vector is pre-processed before training takes place.

19. The method according to claim 2, wherein the at least one heartbeat index includes the highest number of heartbeats within the stipulated duration; the lowest number of heartbeats within the stipulated duration; the average number of heartbeats within the stipulated duration; and a plot of the heartbeat pattern.

20. The system according to claim 8, wherein at least one heartbeat index includes the highest number of heartbeats within the stipulated duration; the lowest number of heartbeats within the stipulated duration; the average number of heartbeats within the stipulated duration; and a plot of the heartbeat pattern.

21. The system according to claim 15, wherein the at least one social sensor comprises a social media account associated with the individual.
